(19) United States
(12) Patent Application Publication
(10) Pub. No.: US 2025/0009702 A1
(43) Pub. Date: Jan. 9, 2025

(54) USE OF CHLOROGENIC ACID IN PREPARATION OF MEDICINE FOR TREATING CENTRAL NERVOUS SYSTEM TUMOR

(71) Applicant: SICHUAN JIUZHANG BIOLOGICAL SCIENCE AND TECHNOLOGY CO., LTD., Chengdu, Sichuan (CN)

(72) Inventors: Jie ZHANG, Chengdu, Sichuan (CN); Wenbin LI, Chengdu, Sichuan (CN); Wang HUANG, Chengdu, Sichuan (CN); Ya ZHANG, Chengdu, Sichuan (CN); Fei ZHANG, Chengdu, Sichuan (CN); Min XU, Chengdu, Sichuan (CN)

(21) Appl. No.: 18/577,383

(22) PCT Filed: Jul. 6, 2022
(86) PCT No.: PCT/CN2022/104224

§ 371 Date: Jan. 8, 2024

(30) Foreign Application Priority Data
Jul. 8, 2021 (CN) 202110775997.8

(51) Int. Cl. A61K 31/216; A61P 35/00

(52) U.S. Cl. CPC A61K 31/216; A61P 35/00

(57) ABSTRACT

Chlorogenic acid is used in the preparation of a medicine for treating a central nervous system tumor. It is discovered that chlorogenic acid may effectively treat diffuse large B-cell lymphoma, which provides a new clinical treatment of central nervous system tumors, especially diffuse large B-cell lymphoma.

### FIELD OF THE INVENTION

The present invention belongs to the field of medicine, and specifically relates to the new use of chlorogenic acid in the preparation of medicaments for treating central nervous system tumors.

### BACKGROUND OF THE INVENTION

Central nervous system lymphoma (CNSL) is a type of tumor that occurs in the central nervous system. Its incidence rate is low, accounting for 1%-3% of central nervous system tumors. With the application of immunosuppressive agents, the incidence rate of the disease has increased in recent years. Central nervous system lymphoma includes the lymphoma originating from the central nervous system and the secondary lymphoma caused by systemic lymphoma invading the central nervous system. The lymphoma originating from the central nervous system, also known as primary central nervous system lymphoma (PCNSL), accounts for approximately 8% of central nervous system lymphoma.

PCNSL is a kind of rare non-Hodgkin lymphoma (NHL) that originates from the central nervous system (including the brain, eyes, spinal cord, meninges, etc.), without other parts being involved. It accounts for about 2% to 4% of all intracranial tumors and 4% to 6% of all non-Hodgkin lymphoma (NHL), and its incidence rate has gradually increased in recent years. Most malignant lymphomas originating from the central nervous system are B-cell non-Hodgkin's lymphoma (B-NHL), which is characterized by diffuse or multifocal. In clinical practice, it often presents with neurological symptoms such as hemiplegia, unstable walking, blurred vision, and may also manifest as increased intracranial pressure (ICP), seizures, personality changes, etc.

Diffuse large B cell lymphoma (DLBCL) is the most common invasive B-cell non-Hodgkin's lymphoma. It is a B-cell-derived medium or highly malignant tumor, and as the most common type of non-Hodgkin's lymphoma (NHL), it accounts for approximately 30% to 40% of adult NHL. Currently, the comprehensive treatment regimen based on high-dose methotrexate (HD-MTX) is used as the primary treatment for DLBCL. It has been found that the combination of chemotherapy and radiotherapy can significantly improve the therapeutic effect on DLBCL, but HD-MTX combined with whole-brain radiotherapy (WBRT) can significantly increase the incidence rate of leukoencephalopathy and cause great long-term neurotoxicity. In addition, clinical results show that now, less than half of patients can be cured by standard chemotherapy regimens, while the rest of patients, even if relieved, may inevitably experience recurrence and death. The main reason for the difficulty in treating DLBCL is the overexpression of multidrug resistance (MDR) genes in cells, which leads to multidrug resistance to chemotherapeutic medicaments. Therefore, there is an urgent need to develop a new drug with low toxicity and side effects, while its therapeutic effects are further improved.

Chlorogenic acid (CHA), also known as caffetannic acid, is an organic acid widely present in plants such as honeysuckle, Eucommia ulmoides leaves (Eucommiaceae), sunflower seeds, Artemisia scoparia, and Scabiosa comosa. It has various pharmacological effects, including anti-oxidant, anti-bacterial, anti-viral, hypoglycemic, hypolipidemic, anti-hypertensive, and immune regulation activities. The Chinese patent application number CN201510079639.8 has disclosed the use of CHA in the manufacturer of medicaments for the prevention and treatment of primary cutaneous T cell lymphoma, in which it has been experimentally proven that CHA can activate CD4T lymphocytes and CD8T lymphocytes, and target CD4T lymphocytes and CD8T lymphocytes to prevent and treat primary cutaneous T-cell lymphoma. However, primary cutaneous T-cell lymphoma is not a central nervous system tumor, and its etiology, location, and treatment mechanism are very different from those of central nervous system tumors. At present, there have been no reports of using CHA to treat central nervous system tumors.

### SUMMARY OF THE INVENTION

The object of the present invention is to provide novel applications of CHA in the manufacturer of medicaments for the prevention and/or treatment of central nervous system tumors.

The present invention provides the use of CHA in the manufacturer of medicaments for the prevention and/or treatment of central nervous system tumors.

Further, the central nervous system tumor is a central nervous system lymphoma.

Further, the central nervous system lymphoma is primary central nervous system lymphoma.

Further, the primary central nervous system lymphoma is B-cell non-Hodgkin's lymphoma (B-NHL).

Further, said B-cell non-Hodgkin's lymphoma is diffuse large B-cell lymphoma.

Further, said diffuse large B-cell lymphoma is that in the brain, eye, or spinal cord.

Further, the medicament is a pharmaceutical preparation made with chlorogenic acid as the active ingredient, in combination with pharmaceutically acceptable excipients.

Further, the pharmaceutical preparation is oral.

Further, the pharmaceutical preparation is injectable.

Further, the unit dosage form of the pharmaceutical formulation contains 0.5-5.5 mg of CHA.

Further, the unit dosage form of the pharmaceutical formulation contains 1.1-3.3 mg of CHA, and preferably 3.3 mg of CHA.

The present invention also provides a medicament for treating central nervous system tumors, which is manufactured with chlorogenic acid as the active ingredient, in combination with pharmaceutically acceptable excipients; the unit dosage form of the medicament contains 0.5-5.5 mg of CHA, preferably 1.1-3.3 mg of CHA, and more preferably 3.3 mg of CHA.

In the present invention, it is first discovered that CHA has therapeutic effect on DLBCL, that provides a new and safe option for the treatment of central nervous system tumors in clinical practice, especially DLBCL, with good application prospects.

Obviously, based on the above content of the present invention, according to the common technical knowledge and the conventional means in the field, other various modifications, alternations, or changes can further be made, without department from the above basic technical spirits.

With reference to the following specific examples, the above content of the present invention is further illustrated. But it should not be construed that the scope of the above subject matter of the present invention is limited to the following examples. The techniques realized based on the above content of the present invention are all within the scope of the present invention.

### DESCRIPTION OF FIGURES

FIG. 1. Growth curves of mice with DLBCL in the brain for each treatment group.

FIG. 2. The tumor inhibition rates of mice in each treatment group.

FIG. 3. Growth curves of mice with DLBCL in the orbit for each treatment group.

FIG. 4. The tumor inhibition rates of mice in each treatment group.

### EXAMPLES

The starting materials and equipment used in the present invention are all known products and can be obtained by purchasing those commercially available.

Example 1 The formula for oral preparation of the present invention

### 1. Formula 1

CHA 1000 g.

Preparation method: CHA was weighed according to the pre-determined amount in the formula, and then aseptically subpacked as powders.

### 2. Formula 2

CHA 1000 g, bulking agent 500 g, binding agent 5 g.

Preparation method: CHA, bulking agent, and binding agent were weighed according to the pre-determined amount in the formula, and then granulated, sieved, and subpacked as granules.

### 3. Formula 3

CHA 1000 g, bulking agent 500 g, binding agent 5 g, and lubricant 3 g.

Preparation method: CHA, bulking agent, and binding agent were weighed according to the pre-determined amount in the formula, and then granulated and sieved, to which was added lubricant, followed by pressing, to obtain tablets.

The above bulking agents were one or more of mannitol, lactose, starch, microcrystalline cellulose, and dextrin; the binding agents were one or two of sodium carboxymethylcellulose and PVP (polyvinylpyrrolidone); the lubricants were one or more of magnesium stearate, talcum powder, and micro silica gel.

Example 2 The formula for CHA injection of the present invention

### 1. Formula 1

CHA 1000 g.

Preparation method (1): CHA was aseptically weighed according to the formula, and aseptically subpacked as powder injection.

Preparative method (2): CHA was weighed according to the formula, dissolved in water for injection, filtered, sterilized, freeze-dried, and filled, to obtain freeze-dried powder injection.

### 2. Formula 2

CHA 1000 g, stent agent 2667 g, and antioxidant 67 g.

Preparation method: CHA, stent agent, and antioxidant were weighed according to the formula, dissolved in water for injection, filtered, sterilized, filled, and freeze-dried (Freeze drying conditions: Pre-freezing: temperature≤−40° C., normal pressure, drying time 2-4 h, freezing; the first drying: temperature≤−13° C., negative pressure, drying time ≥12 h, fully dry; the secondary drying, temperature 20-30° C., negative pressure, drying time ≥2 h), to obtain freeze-dried powder injection.

Said stent agents were one or more of mannitol, lactose and glucose; the antioxidants were one or more of sodium bisulfite, vitamin, glutathione, and folic acid.

The beneficial effects of the present invention were demonstrated by experimental examples.

### Experiment Example 1 In Vitro Experiment on Treatment of Central Nervous System Lymphoma with CHA

### 1. Experimental Materials

### 1.1 Test Lymphoma Cell Lines

The diffuse large B-cell lymphoma cell lines pfeiffer was purchased from ATCC (USA), and passaged in RPMI 1640 medium.

### 1.2 Test Drugs

Positive drug: methotrexate for injection, provided by Jiangsu Hengrui Pharmaceutical Co., Ltd.

Test drug: chlorogenic acid raw material.

Both above drugs were prepared and diluted with serum-free RPMI 1640 medium to the expected concentration, and then filtered by a 0.22 μm microporous filter membrane for sterilization. They were stored in a refrigerator at 4° C. for later use.

1.3 RPML1640 medium

RPML1640 cell culture medium was dissolved in ultrapure water (1000 ml) under stirring, to which were added 2.2 g of NaHCO₃ and 10 ml of HEPES, and then the solution was stirred to dissolve, followed by addition of suitable amounts of penicillin (with a final concentration of 100 U/ml) and streptomycin (with a final concentration of 100 μg/ml). The resultant solution was well mixed, aseptically filtered over 0.22 μm filter membrane, subpackaged, and cryopreserved at −20° C., to obtain the basic medium.

### 2. Experimental Method

### 2.1 Cytology Experiments

### 2.1.1 Cell Recovery

The cryogenic vials containing cells were thawed in a 37° C. water bath, and shaken quickly to melt as soon as possible. The melted cell suspension was moved into a 10 ml centrifuge tube, and centrifuged at a low speed of 1000 r/min for 5-10 min. The supernatant was discarded, followed by addition of a suitable amount of fresh medium. The cells were gently blown, mixed well, and centrifuged again (1000 r/min) for 5 min, to minimize DMSO in the medium. After the cells were additionally centrifuged to discard the supernatant, fresh medium and 10% FBS were added, and then the cells and the medium were blown to mix well, that was placed in a culture flask and then cultured in a 5% CO₂ incubator at a constant temperature of 37° C.

### 2.1.2 Cell Culture Methods

The color of the cell medium was observed daily, together with detection of the cell morphology under a microscope, and according to the cell state, the medium was changed or the cells were passed on every 1-2 days, to maintain a cell growth density of 4-6×10⁵/ml. When the color of the medium became yellow from red, indicating the medium needed to be exchanged, the cells were placed in a 50 ml centrifuge tube, and centrifuged at a low speed of 1000 r/min for 5-10 min, to discard the supernatant, followed by addition of fresh medium. The cell suspension was gently blown, allowed the cells to mix well with the medium, and placed in the culture bottle again. As cells grew, their density increased, and they needed to be passaged. Half of the volume of cell suspension was transferred to another culture flask, and then fresh medium was added to the original volume.

### 2.1.3 Cell Cryopreservation

Pfeiffer cells in logarithmic growth phase were collected and centrifuged at a low speed of 1000 r/min for 5-10 min, to discard the supernatant. The cell precipitate was resuspended with cryopreservation solution, and then the cells were counted and further diluted with cryopreservation solution, to achieve a cell density of 1×10⁶-5×10⁶/mL. The cell suspension was divided into different store tubes, followed by labelling the cell name and cryopreservation date. The tubes were placed in a refrigerator at +4° C. for more than 2 hours, a refrigerator at −20° C. for more than 24 hours, and a refrigerator at −70° C. for storage or stored in a liquid nitrogen tank at −196° C. for a long time.

### 2.2 MTS Assay of the In Vitro Inhibitory Effect of the Treatment Group on DLBCL Cells (Pfeiffer)

### 2.2.1 Experimental Groups

This experiment included 8 groups: blank group, negative control group, positive control group, and 5 test drug groups.

Blank group: only adding the medium, without inoculating pfeiffer cells; Negative control group: adding the medium and inoculating pfeiffer cells; Positive control group: cultivating pfeiffer cells in the presence of 10 μg/ml methotrexate; Test drug group: The test drug CHA was cultured with pfeiffer cells at the concentration of 5 μg/ml, 10 μg/ml, 30 μg/ml, 50 μg/ml, and 100 μg/ml, respectively.

### 2.2.2 Cell Inoculation

The cells in logarithmic growth phase were digested with 0.25% trypsin, washed with the basic medium, and centrifuged (1000 r/min, twice in 5 minutes). The cells were suspended with complete medium, and the cell concentration was adjusted to 6×10⁴/ml, which was inoculated into a 96-well plate at 50 μl/well (6×10³ cells/well).

### 2.3 Administration

The next day, after the cells adhered to the wall, the drug was given to each group and then incubated for 48 h at a constant temperature of 37° C. under 5% CO₂. The dosage for each well is 100 μl. For the blank group, no cell was inoculated, and an equal amount of medium (100 μl) was added; the negative control group received an equal amount of medium (100 μl) after cell inoculation, and 3 wells were set for one group.

### 2.4 Staining

20 μl of MTS was added to each well. The plate was further cultivated for 3 h for color development, and then shaken for 10 seconds before testing, to mix well.

### 2.5 Determination

The OD value was determined at a wavelength of 490 nm using an enzyme-linked immunosorbent assay (ELISA), and the OD value of the blank control was adjusted to 0.

### 2.6 Data Processing

The growth inhibition rate of the tumor cells was calculated according to the following formula:

GI (Growth inhibition rate)=(1−OD_{treatment group}/OD_{negative control group})×100%.

### 3. Experimental Results

### 3.1 Observation Results of Cell Morphology

After each group was administered and treated for 48 h, the cell morphology was observed under an inverted microscope. Compared with the negative control group, the positive control group and the CHA groups had a certain impact on cell growth, with some cells becoming round, shedding, and suspending; among them, the CHA treatment group had a significant inhibitory effect on the growth of pfeiffer cancer cell lines at a concentration of 50-100 μg/ml, with many cells becoming round, shedding, and suspending.

### 3.2 the Inhibition Rate and Dose-Response Curve of Each Treatment Group Against Pfeiffer Cancer Cells

The inhibition rates of each treatment group on pfeiffer cancer cells are shown in Table 1.

TABLE 1
The inhibition rates of each treatment
group on pfeiffer cancer cells.

| | Concentration | OD value | | | | Inhibition |
|---|---|---|---|---|---|---|
| Drugs | (μg/ml) | 1 | 2 | 3 | Mean | rate (%) |
| Negative | — | 2.322 | 2.317 | 2.335 | 2.325 | — |
| control | | | | | | |
| group | | | | | | |
| Positive | 10 | 1.202 | 1.193 | 1.185 | 1.193 | 48.67% |
| control | | | | | | |
| group | | | | | | |
| CHA treatment | 5 | 1.335 | 1.221 | 1.233 | 1.263 | 45.68% |
| group | 10 | 1.156 | 1.142 | 1.15 | 1.149 | 50.57% |
| | 30 | 1.006 | 1.007 | 0.98 | 0.998 | 57.09% |
| | 50 | 0.630 | 0.621 | 0.634 | 0.628 | 72.97% |
| | 100 | 0.661 | 0.65 | 0.664 | 0.658 | 71.68% |

As shown in the above table, the CHA treatment group had a significant inhibitory effect on pfeiffer cancer cells, and when the concentration of CHA was 50-100 μg/ml, the tumor inhibition rate was significant, up to 71.68-72.97%; in addition, the same dose (10 μg/ml) of CHA has a higher inhibitory effect on pfeiffer cancer cells than the positive control drug methotrexate.

The above experimental results indicated that CHA had a significant in vitro inhibitory effect on DLBCL pfeiffer cells, and in this system, the tumor inhibition action was more significant when the concentration of chlorogenic acid was 50-100 μg/ml.

### Experiment Example 2: In Vivo Animal Experiment on Treatment of DLBCL in the Brain with CHA

### 1. Experimental Materials

### 1.1 Test Drugs

Test drug 1: CHA for injection, the formula ratio: CHA, mannitol, sodium bisulfite (30:80:2);

Test drug 2: CHA oral preparation, the formula ratio: CHA, bulking agent (lactose), binding agent (sodium carboxymethyl cellulose) (1000:500:5);

Positive drug: methotrexate, Jiangsu Hengrui Pharmaceutical Co., Ltd.

For the above CHA preparation for injection, CHA, mannitol, and sodium bisulfite were weighed according to the pre-determined amount in the formula, dissolved in water for injection, filtered, sterilized, filled, and freeze-dried (Freeze drying conditions: Pre-freezing: temperature≤−40° C., normal pressure, drying time 2-4 h, freezing; the first drying: temperature≤−13° C., negative pressure, drying time ≥12 h, fully dry; the secondary drying, temperature 20-30° C., negative pressure, drying time ≥2 h), to obtain freeze-dried powder injection with a labeled amount of 30 mg/vial of CHA.

For the above CHA oral preparation, CHA, lactose, and sodium carboxymethyl cellulose were weighed according to the pre-determined amount in the formula, and then granulated, sieved, and subpacked as granules.

1.2 Test cell lines

DLBCL Ly8 cell lines were purchased from the Cancer Hospital of Fudan University, which were suspended, cultured, and passaged in 10% calf serum DMEM complete medium, with stable growth. Cells in the logarithmic growth phase were collected, centrifuged, counted, and diluted to 10⁴/μl for later use. Cells were inoculated at 2×10⁴ cells/animal.

### 1.3 Test Animals

BALB/c mice: 60 mice, half male and half female, 3-4 weeks old, weighing 15-22 g, purchased from the Experimental Animal Management Center of West China Medical Center. The breeding room was kept at a constant temperature, clean, and regularly disinfected. Replacement of tools, bedding, and drinking water was all carried out under sterile conditions.

### 2. Experimental Methods

### 2.1 Establishment of a DLBCL Model in the Brain of Experimental Animals

The mice were anesthetized by intraperitoneal injection of 1% pentobarbital sodium (0.2 μl), fixed on a stereotactic device, and disinfected with 2% chlorhexidine iodine solution on the skin of skull part. The injection site of the syringe was 0.1 cm to the left or right of the midline of the skull, and 0.3 cm in front of the coronal suture in mice. The microsyringe was sequentially penetrated through the skin and skull of the mouse skull part, and was fixed about 1-2 mm into the skull. 2 μl of Ly8 cell suspension was slowly injected (about 2×10⁴ cells), and the injection was performed within 15 minutes, followed by leaving the needle for 5 minutes to allow the cells to fully attach, and then slowly removing the needle. There is no need to inject antibiotics after surgery. During sleep after anesthesia, the mice were kept at 30° C. for 2 h. After anesthesia wore off, the mice were placed at room temperature of 25° C., and the surface changes were observed 24 h after inoculation. The following symptoms were observed: topical protrusion on the injection side, body emaciation, lethargy, and decreased mobility.

### 2.2 Experimental Groups

The successfully inoculated mice were randomly divided into 7 groups, with 6 mice in each group, including:
(1) Negative control group: Intraperitoneal injection of physiological saline, once a day;
(2) Positive drug group: Intraperitoneal injection of methotrexate, once every two days, 3 mg/kg/time;
(3) CHA oral granule group: Oral administration of test drug 2, once a day, 30 mg/kg/time;
(4) Injection treatment group 1: Intraperitoneal injection of test drug 1, once a day, 5 mg/kg/time;
(5) Injection treatment group 2: Intraperitoneal injection of test drug 1, once a day, 10 mg/kg/time;
(6) Injection treatment group 3: Intraperitoneal injection of test drug 1, once a day, 30 mg/kg/time;
(7) Injection treatment group 4: Intraperitoneal injection of test drug 1, once a day, 50 mg/kg/time;

The mice in the above test drug groups receiving the prepared drug were administered by intraperitoneal injection at 0.2 mL/10 g body weight. The drug preparation was formulated prior to daily administration, and given for 14 days.

### 2.3 Evaluation of Therapeutic Effects

During the administration period, the growth of the mouse tumor, eating, activity, and other adverse reactions were observed and recorded every other day. After 14 days of administration, the mice were euthanized using cervical dislocation method, and their body weight and tumor size were measured and recorded according to the pre-administration numbers. The tumor inhibition rate (%) was calculated based on tumor weight. The body weight and tumor weight of mice were expressed as mean±standard deviation (X̅±SD), and t-tests were performed between each treatment group and negative control group, as well as between each treatment group and positive control methotrexate group.

$$The tumor inhibition rate = \frac{\begin{matrix}The tumor weight of negative control group - \\ The tumor weight of each treatment group\end{matrix}}{The tumor weight of negative control group} \times 100 \% .$$

### 3. Experimental Results

### 3.1 Establishment of Models in Experimental Animals

60 BALB/c mice were inoculated with Ly8 cells to establish a transplanted tumor model. In the initial stage, a localized protrusion was observed at the inoculation site, which disappeared after 2-3 days. After about 5 days, a small protrusion began to grow at the inoculation site of some BALB/c mice; after about 14 days, the protrusion in this area gradually grew, and the maximum diameter of the transplanted tumor grew to about 5 mm. A total of 45 BALB/c mice were successfully established as transplant tumor models, which continued to grow. The success rate of inoculation was 75%.

### 3.2 Therapeutic Effects of Each Experimental Group on DLBCL in the Brain

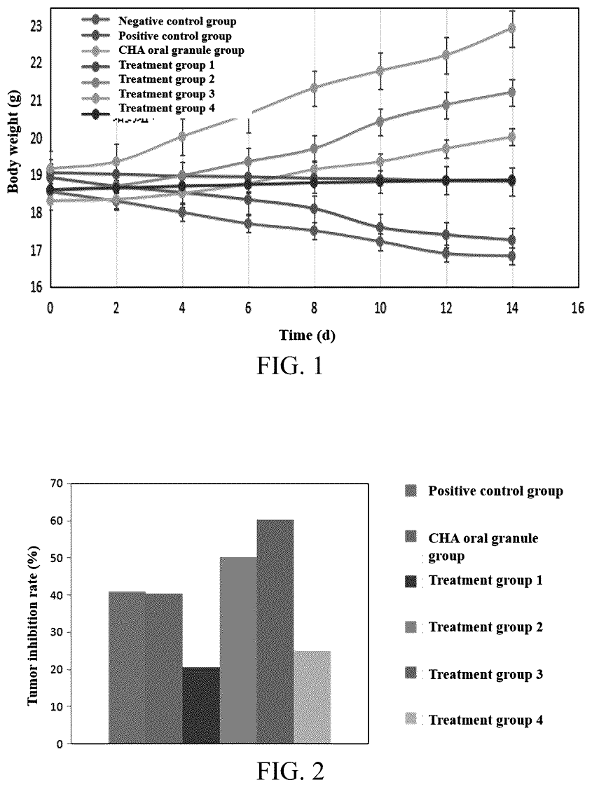

After successful inoculation, 42 mice were selected and randomly divided into 7 groups. The mice were administered according to the experimental regime, and the changes in the body weight and tumor weight were recorded. The results are shown in Table 2 as well as FIGS. 1 and 2.

TABLE 2
Observation on treatment of mice in each group.

| | | Weight (g) | | | Tumor | Survival |
|---|---|---|---|---|---|---|
| | Dosage | Before | After | Tumor | inhibition | number |
| Groups | (mg · kg⁻¹) | treatment | treatment | weight (g) | rate (%) | (mouse) |
| Negative | — | 18.55 ± 1.02 | 16.83 ± 1.33 | 1.51 ± 0.16 | — | 4 |
| Positive | 3 | 18.93 ± 0.87 | 17.26 ± 0.65 | 0.79 ± 0.12* | 41.06 | 6 |
| CHA oral | 30 | 18.31 ± 0.70 | 20.02 ± 1.06* | 0.90 ± 0.08* | 40.40 | 6 |
| Treatment | 5 | 19.06 ± 1.12 | 18.83 ± 1.15 | 1.20 ± 0.09 | 20.53 | 6 |
| Treatment | 10 | 18.57 ± 0.52 | 21.22 ± 1.01* | 0.75 ± 0.10* | 50.33 | 7 |
| Treatment | 30 | 19.17 ± 1.04 | 22.93 ± 0.84* | 0.60 ± 0.11*^{#} | 60.26 | 7 |
| Treatment | 50 | 18.61 ± 0.83 | 18.87 ± 0.75 | 1.13 ± 0.16 | 25.17 | 6 |

Note:
*P < 0.01, compared to the negative control group;
^{#}P < 0.05, compared to the positive control group.

As shown in Table 2 as well as FIGS. 1 and 2, the followings could be found:
(1) The positive drug group, 30 mg/kg CHA oral granule group, and 10-30 mg/kg treatment groups of CHA injection had good therapeutic effects on DLBCL in the brain, with significant differences compared to the negative control group (P<0.05).
(2) There were 3 deaths in the negative control group, and 1 death in the positive control group, oral granule group, and 5 mg/kg and 50 mg/kg injection treatment groups, as well as no death in 10-30 mg/kg injection treatment groups, which indicated that the effective dose group of CHA injection had a good therapeutic effect; in addition, by close observation, the mice of the negative control group showed symptoms such as lack of luster in their fur, loss of appetite, and decreased activity. As the tumor load increased, the above symptoms became increasingly apparent. The symptoms of the mice in the effective dose treatment group improved significantly, and there were no adverse reactions related to treatment medication.
(3) The growth curves of mice in the oral CHA granule group and the effective dose groups of CHA injection showed a gradual upward trend, while the negative control group and positive control group showed a downward trend. The changes in the injection groups of 5 mg/kg and 50 mg/kg were not significant, indicating that the effective dose group of CHA injection and the oral CHA granule group had a certain promoting effect on the growth and development of diseased mice.
(4) There was a significant difference in tumor weight between the 10 mg/kg-30 mg/kg injection group and the positive control group (P<0.05). Among them, at a dosage of 30 mg/kg, the CHA injection had the best therapeutic effect on DLBCL in the brain, indicating that 30 mg/kg was the optimal dosage.

The above experimental results indicated that CHA had a good in vivo therapeutic effect on DLBCL in the brain, in which the injection doses ranging from 10 mg/kg to 30 mg/kg had a higher tumor inhibition rate. The best dosage was 30 mg/kg.

According to the Methodology of Pharmacological Experiment by Shu-Yun Xu et al., the dose in mice is equivalent to 9.1 times that in humans, calculated as a unit weight dose. Therefore, it could be concluded that the best therapeutic effect for patients with DLBCL in the brain was achieved when CHA was injected into the human body at a dosage of 3.3 mg/kg.

### Experiment Example 3: In Vivo Animal Experiment on Treatment of DLBCL in the Orbit with Cha

### 1. Experimental Materials

### 1.1 Test Drugs

Test drug 1: CHA for injection, formula ratio: CHA, mannitol, sodium bisulfite (30:80:2);

Test drug 2: CHA oral preparation, formula ratio: CHA, bulking agent (lactose), binding agent (sodium carboxymethyl cellulose) (1000:500:5);

Positive drug: methotrexate, Jiangsu Hengrui Pharmaceutical Co., Ltd.

CHA 1000 g, stent agent 2667 g, and antioxidant 67 g.

For the above CHA preparation for injection, CHA, mannitol, and sodium bisulfite were weighed according to the pre-determined amount in the formula, dissolved in water for injection, filtered, sterilized, filled, and freeze-dried (Freeze drying conditions: Pre-freezing: temperature≤−40° C., normal pressure, drying time 2-4 h, freezing; the first drying: temperature≤−13° C., negative pressure, drying time ≥12 h, fully dry; the secondary drying, temperature 20-30° C., negative pressure, drying time ≥2 h), to obtain freeze-dried powder injection with a labeled amount of 30 mg/vial of CHA.

For the above CHA oral preparation, CHA, lactose, and sodium carboxymethyl cellulose were weighed according to the pre-determined amount in the formula, and then granulated, sieved, and subpacked as granules.

### 1.2 Test Cell Lines

DLBCL pfeiffercell lines were purchased from ATCC, USA.

### 1.3 Test Animals

BALB/c mice: 60 mice, half male and half female, 3-4 weeks old, weighing 15-22 g, purchased from the Experimental Animal Management Center of West China Medical Center. The breeding room was kept at a constant temperature, clean, and regularly disinfected. Replacement of tools, bedding, and drinking water was all carried out under sterile conditions.

### 2. Experimental Methods

### 2.1 Preparation of Pfeiffer Cells

The cells were thawed in a 37° C. water bath according to the method provided by ATCC, and cultured in RPMI-1640 medium. The medium was exchanged or the cells were passaged every 1-2 days. When the cells were in a stable proliferative state, the cells with good vitality were selected from the 2nd to 3rd generations and counted under a microscope using a cell counting board. The concentration of inoculate cells was adjusted to 1.5×10⁸/ml, and sealed for later use.

### 2.2 Establishment of a DLBCL Model in the Orbit of Experimental Animals

The tumor cell suspension inoculation method was used. Before cell inoculation, all mice were subjected to cesium-137 radiation to further immunosuppress and improve the success rate of inoculation. Tumor cells were inoculated within 6 hours after irradiation. After disinfecting with 75% alcohol around the eyes, 0.1 ml of tumor cell suspension was drawn using an 1 ml empty needle, and injected into the orbit through the skin of the upper temporal eyelid. After removing the needle, the injection site was pressed for 5 min, and no obvious abnormalities were observed in the nude mice. After inoculation, high-pressure sterilized water containing 100000 U/L of gentamicin was given for one week to prevent infection. 5% glucose with egg yolk was orally administered daily to enhance nutrition. The growth state of mice and local tumor growth was observed every day.

### 2.3 Experimental Groups

After successful inoculation, 42 mice were randomly divided into 7 groups, with 6 mice in each group, including:
(1) Negative control group: Intraperitoneal injection of physiological saline, once a day;
(2) Positive drug group: Intraperitoneal injection of methotrexate, once every two days, 3 mg/kg/time;
(3) CHA oral granule group: Oral administration of test drug 2, once a day, 30 mg/kg/time;
(4) Injection treatment group 1: Intraperitoneal injection of test drug 1, once a day, 5 mg/kg/time;
(5) Injection treatment group 2: Intraperitoneal injection of test drug 1, once a day, 10 mg/kg/time;
(6) Injection treatment group 3: Intraperitoneal injection of test drug 1, once a day, 30 mg/kg/time;
(7) Injection treatment group 4: Intraperitoneal injection of test drug 1, once a day, 50 mg/kg/time; The mice in the above test drug groups receiving the prepared drug were administered by intraperitoneal injection at 0.2 mL/10 g body weight. The drug preparation was formulated prior to daily administration, and given for 14 days.

### 2.4 Evaluation of Therapeutic Effects

During the administration period, the growth of the mouse tumor, eating, activity, and other adverse reactions were observed and recorded every other day. After 14 days of administration, the mice were euthanized using cervical dislocation method, and their body weight and tumor size were measured and recorded according to the pre-administration numbers. The tumor inhibition rate (%) was calculated based on tumor weight. The body weight and tumor weight of mice were expressed as mean±standard deviation (X̅±SD), and t-tests were performed between each treatment group and negative control group, as well as between each treatment group and positive control methotrexate group.

$$The tumor inhibition rate = \frac{\begin{matrix}The tumor weight of negative control group - \\ The tumor weight of each treatment group\end{matrix}}{The tumor weight of negative control group} \times 100 \% .$$

### 3. Experimental Results

### 3.1 Establishment of Models in Experimental Animals

After inoculation in the right orbit of mice, slight ocular proptosis was observed, which returned to normal on the second day. On the 12th day, 40 BALB/C mice exhibited eye symptoms, such as inability to open the eyelids, tearing, mild edema of the conjunctiva around the eyeball, and no proptosis; on the 15th day, 55 BALB/C nude mice showed eye symptoms, including tearing, obvious swelling of the conjunctiva around the eyeballs, and no obvious protrusion. The success rate of inoculation was 91.67%.

### 3.2 Therapeutic Effects of Each Experimental Group on DLBCL in the Orbit

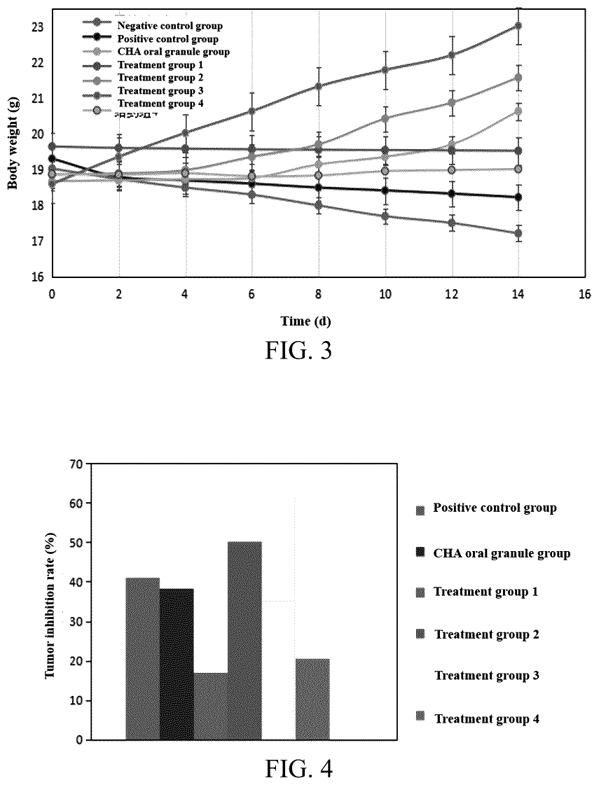

After successful inoculation, 42 mice were selected and randomly divided into 7 groups. The mice were administered according to the experimental regime, and the changes in the body weight and tumor weight were recorded. The results are shown in Table 3 as well as FIGS. 3 and 4.

TABLE 3
Observation on treatment of mice in each group.

| | | | | | Tumor | |
|---|---|---|---|---|---|---|
| | | Body weight (g) | | Tumor | inhibition | Survival |
| | Dosage | Before | After | weight | rate | number |
| Groups | (mg · kg⁻¹) | administration | administration | (g) | (%) | (mice) |
| Negative | — | 19.03 ± 0.98 | 17.22 ± 0.85 | 1.75 ± 0.09 | — | 5 |
| control group | | | | | | |
| Positive control | 3 | 19.31 ± 0.66 | 18.23 ± 0.79 | 1.03 ± 0.10* | 41.14 | 6 |
| group | | | | | | |
| CHA oral | 30 | 18.68 ± 0.91 | 20.64 ± 1.15* | 1.08 ± 0.09* | 38.29 | 6 |
| granule group | | | | | | |
| Treatment | 5 | 19.65 ± 1.27 | 19.53 ± 1.18 | 1.45 ± 0.08 | 17.14 | 6 |
| group 1 | | | | | | |
| Treatment | 10 | 18.86 ± 0.93 | 21.58 ± 1.09* | 0.87 ± 0.12*^{#} | 50.29 | 7 |
| group 2 | | | | | | |
| Treatment | 30 | 18.60 ± 1.10 | 23.03 ± 0.96* | 0.66 ± 0.10*^{#} | 62.29 | 7 |
| group 3 | | | | | | |
| Treatment | 50 | 18.88 ± 0.80 | 19.02 ± 0.87 | 1.39 ± 0.11 | 20.57 | 6 |
| group 4 | | | | | | |

Note:
*P < 0.01, compared to the negative control group;
^{#}P < 0.05, compared to the positive control group.

As shown in Table 3 as well as FIGS. 3 and 4, the followings could be concluded:
(1) The positive drug group, 30 mg/kg CHA oral granule group, and 10-30 mg/kg treatment groups of CHA injection had good therapeutic effects on DLBCL in the orbit, with significant differences compared to the negative control group (P<0.05).
(2) There were 2 deaths in the negative control group, and 1 death in the positive control group, oral granule group, and 5 mg/kg and 50 mg/kg injection treatment groups, as well as no deaths in 10-30 mg/kg injection treatment groups, which indicated that the effective dose group of CHA injection had a good therapeutic effect; in addition, by close observation, as the time went, the negative control group gradually became more prominent, and the tumor gradually wrapped the eyeball, that would ultimately cause the eyelids to be unable to close and the eyeball to be covered, and then the tumor was exposed to the outside, accompanied by bleeding and increased tearing. The mice in the CHA oral granule group and the injection groups of 10-30 mg/kg showed significant improvement in eye discomfort, reduced abscess, and relieved tearing symptoms.
(3) The growth curves of mice in the oral CHA granule group and the effective dose groups of CHA injection showed a gradual upward trend, while the negative control group and positive control group showed a downward trend. The changes in the injection groups of 5 mg/kg and 50 mg/kg were not significant, indicating that the effective dose group of CHA injection and the oral CHA granule group had a certain promoting effect on the growth and development of diseased mice.
(4) There was a significant difference in tumor weight between the 10 mg/kg-30 mg/kg injection group and the positive control group (P<0.05). Among them, at a dosage of 30 mg/kg, the CHA injection had the best therapeutic effect on DLBCL in the orbit, indicating that 30 mg/kg was the optimal dosage.

The above experimental results indicated that CHA had a good in vivo therapeutic effect on DLBCL in the orbit, in which the injection doses ranging from 10 mg/kg to 30 mg/kg had a higher tumor inhibition rate. The best dosage was 30 mg/kg. It can be concluded that the best treatment effect for patients with DLBCL in the orbit was achieved when CHA was injected into the human body at a dosage of 3.3 mg/kg.

In summary, the present invention provided the use of CHA in the manufacturer of a medicament for the prevention and/or treatment of central nervous system tumors. Additionally, in the present invention, it was first discovered that CHA has therapeutic effect on DLBCL, that provided a new and safe option in clinical practice for the treatment of central nervous system tumors, especially DLBCL, with good application prospects.

1. Chlorogenic acid for use in the manufacturer of medicaments for the prevention and/or treatment of central nervous system tumors.

2. The use according to claim 1, characterized in that the central nervous system tumor is a central nervous system lymphoma.

3. The use according to claim 2, characterized in that the central nervous system lymphoma is primary central nervous system lymphoma.

4. The use according to claim 3, characterized in that the primary central nervous system lymphoma is B-cell non-Hodgkin's lymphoma (B-NHL).

5. The use according to claim 4, characterized in that said B-cell non-Hodgkin's lymphoma is diffuse large B-cell lymphoma (DLBCL).

6. The use according to claim 5, characterized in that said diffuse large B-cell lymphoma is that in the brain, eye, or spinal cord.

7. The use according to claim 1, characterized in that the medicament is a pharmaceutical preparation made with chlorogenic acid as the active ingredient, in combination with pharmaceutically acceptable excipients.

8. The use according to claim 7, characterized in that the pharmaceutical preparation is oral or injectable.

9. The use according to claim 8, characterized in that the unit dosage form of the pharmaceutical formulation contains 0.5-5.5 mg of chlorogenic acid, preferably 1.1-3.3 mg of chlorogenic acid, and more preferably 3.3 mg of chlorogenic acid.

10. A medicament for treating central nervous system tumors, which is manufactured with chlorogenic acid as the active ingredient, in combination with pharmaceutically acceptable excipients; the unit dosage form of the medicament contains 0.5-5.5 mg of chlorogenic acid, preferably 1.1-3.3 mg of chlorogenic acid, and more preferably 3.3 mg of chlorogenic acid.
